(19) United States
(12) Patent Application Publication
(10) Pub. No.: US 2007/0196522 A1
(43) Pub. Date: Aug. 23, 2007

(54) Methods and compositions for nerve regeneration

(75) Inventors: Amala Soumyanath, Portland, OR (US); Bruce G. Gold, West Linn, OR (US); Sandra Gold, West Linn, OR (US); Yong-Ping Zhong, Beaverton, OR (US); Dennis Bourdette, Portland, OR (US)

Correspondence Address: KLARQUIST SPARKMAN, LLP, 121 SW SALMON STREET, SUITE 1600, PORTLAND, OR, 97204, US

(73) Assignee: Oregon Health and Science University

(21) Appl. No.: 11/644,690

(22) Filed: Dec. 22, 2006

Related U.S. Application Data
(63) Continuation-in-part of application No. PCT/US05/21150, filed on Jun. 14, 2005.
(60) Provisional application No. 60/584,408, filed on Jun. 29, 2004.
(60) Provisional application No. 60/588,602, filed on Jul. 16, 2004.
(60) Provisional application No. 60/616,072, filed on Oct. 4, 2004.

(51) Int. Cl. A61K 36/28

(52) U.S. Cl. 424/764

(57) ABSTRACT

A method of promoting nerve regeneration in a subject that includes administering to the subject a therapeutically effective amount of a composition that includes at least one therapeutically-active extract fraction of Centella asiatica. One example of making the extract fraction of Centella asiatica involves extracting Centella asiatica plant material resulting in an extract residue and successively fractionating the Centella asiatica extract residue with at least two eluants of increasing polarity.

This application is a continuation-in-part of International Application No. PCT/US2005/021150, filed Jun. 14, 2005, and designating the United States, which, in turn, claims the benefit of U.S. Provisional Application No. 60/584,408, filed Jun. 29, 2004, U.S. Provisional Application No. 60/588,602, filed Jul. 16, 2004, and U.S. Provisional Application No. 60/616,072, filed Oct. 4, 2004, all of which are incorporated herein by reference in their entireties.

### FIELD

The present disclosure relates to methods and compositions for promoting nerve regeneration.

### BACKGROUND

Nerve regeneration in the peripheral nervous system occurs in accordance with the following processes: first, Schwann cells are separated from cut axons to obtain division potential (dedifferentiation), axons of nerve cells regrow from injured sites, Schwann cells insulate the re-grown axons with myelin sheaths (redifferentiation), and then axons grow enough to reach targets such as muscles to form neuromuscular junctions at muscle cells.

Following traumatic or disease-induced axonal degeneration or transection in the peripheral nervous system, axonal regeneration and neural network re-connectivity may ensue, often resulting in at least partial functional recovery. Within the peripheral nervous system, this cellular regenerative property of neurons has limited ability to repair function to a damaged neural pathway. Specifically, the new axons extend randomly, and are often misdirected, making contact with inappropriate targets that can cause abnormal function. For example, if a motor nerve is damaged, regrowing axons may contact the wrong muscles, resulting in paralysis. In addition, where severed nerve processes result in a gap of longer than a few millimeters, e.g., greater than 10 millimeters (mm), appropriate nerve regeneration does not occur, either because the processes fail to grow the necessary distance, or because of misdirected axonal growth. Moreover, the rate of axonal elongation (3-4 mm/day) is slow. Consequently, recovery is measured in weeks or months, depending upon the distance between the site of injury and the target tissue. Therapies that speed regeneration over long distances would be highly beneficial to patients and would significantly reduce health care costs.

Mammalian neural pathways also are at risk due to damage caused by neoplastic lesions. Neoplasias of both the neurons and glial cells have been identified. Transformed cells of neural origin generally lose their ability to behave as normal differentiated cells and can destroy neural pathways by loss of function. In addition, the proliferating tumors may induce lesions by distorting normal nerve tissue structure, inhibiting pathways by compressing nerves, inhibiting cerebrospinal fluid or blood supply flow, and/or by stimulating the body's immune response. Metastatic tumors, which are a significant cause of neoplastic lesions in the brain and spinal cord, also similarly may damage neural pathways and induce neuronal cell death.

In addition, many of the compounds previously shown to stimulate nerve regeneration have undesired side-effects, such as immunosuppression (FK506 and analogs that retain immunosuppressant activity) or androgenic or estrogenic stimulation. There is therefore a need to provide a class of nerve regeneration compounds that are well tolerated by subjects who take them.

Centella asiatica herb (e.g., Centella asiatica (L.) Urban (Umbelliferae) syn Hydrocotyle asiatica L.), commonly known as “gotu kola”, is an Indian medicinal plant (Indian Pennywort) used for over 2000 years. Modern uses include treatment of psoriasis, skin ulcers, wound healing, leprosy, and as a general “nerve tonic” and memory booster. For example, extracts of Centella asiatica have been used in traditional medicine as a “stimulatory-nervine tonic.” Veerenda Kumar et al., Journal of Ethnopharmacology 79:253-60 (2002). Extracts of Centella asiatica are also commercially available. Asiatic acid, asiaticoside, madecassic acid, and madecassoside are known triterpenoid compounds that are present in Centella asiatica extracts. Kartnig, Clinical Applications of Centella asiatica (L.) Urb., In: L. E. Craker, J. E. Simon (Eds.) Recent advances in botany, horticulture and pharmacology. Herbs Spices Med Plants 3:145-173, 1988. However, there is no knowledge of specific components or compounds of Centella asiatica extracts that have activity specifically for nerve regeneration.

### SUMMARY

Disclosed herein are several methods for promoting nerve regeneration that involve administering at least one extract of Centella asiatica.

In one aspect, there is described a method of promoting nerve regeneration in a subject that includes administering to the subject a therapeutically effective amount of a composition that includes at least one therapeutically-active extract fraction of Centella asiatica.

Another aspect is a method of promoting nerve regeneration of at least one partially or fully transected nerve in a mammal that includes administering to the mammal a therapeutically effective amount of a composition that includes at least one therapeutically-active, substantially apolar extract fraction of Centella asiatica.

An additional aspect is a method of promoting nerve regeneration of at least one partially or fully transected nerve of a peripheral nervous system of a mammal that includes administering to the mammal a therapeutically effective amount of an extract of Centella asiatica.

It has also been found that one especially effective component of the Centella asiatica extract for nerve regeneration is asiatic acid. Asiaticoside and madecassic acid have also been found to have bioactivity for nerve regeneration.

Also disclosed herein is a method for making an extract fraction of Centella asiatica that includes extracting dried Centella asiatica plant material resulting in an extract residue; and successively fractionating the Centella asiatica extract residue with at least two eluants of increasing polarity.

Pharmaceutical compositions that include extract fractions of Centella asiatica are also described herein. In one embodiment, there is disclosed a pharmaceutical composition comprising at least one extract fraction of Centella asiatica, wherein the extract fraction comprises at least about 0.5 dry wt. % of nerve regeneration-active compounds.

According to another approach described herein, nerve regeneration of at least one partially or fully transected nerve of the peripheral nervous system may be promoted via administration of Centella asiatica plant material that is provided in the form of a tablet or capsule.

According to a further embodiment, described herein is a method for providing anti-oxidant neuroprotection in a subject that includes administering to the subject a therapeutically effective amount of a composition that includes at least one therapeutically-active extract fraction of Centella asiatica.

### BRIEF DESCRIPTION OF THE FIGURES

FIG. 1 depicts the results of a high performance liquid chromatography analysis (HPLC) of an ethanolic extract of Centella asiatica (labeled “GK2” as described below), an aqueous extract of Centella asiatica (labeled as “GKW” as described below), and asiatic acid (AA). HPLC analysis was performed using an Econosil 5μ C18 column (250 mm×4.6 mm), eluting with a water:acetonitrile gradient containing 1% acetic acid, detection wavelength 205 nm.

FIG. 2 depicts the results of thin layer chromatography (TLC) analysis of an ethanolic extract of Centella asiatica (labeled “GK2” as described below), an aqueous extract of Centella asiatica (labeled as “GKW” as described below), and asiatic acid (AA). TLC analysis was performed on Silica gel 0.25 mm plates, developing first with chloroform:methanol:water 85:15:1 to SF1, and then with chloroform:glacial acetic acid: methanol: water 60:32:25:10.5 to SF2. The plate was sprayed with anisaldehyde:sulphuric acid reagent (Wagner and Bladt, 1996, Plant Drug Analysis. A thin layer chromatography atlas. 2nd Ed. Springer-Verlag, Berlin Heidelberg, p. 307).

FIGS. 3-10 are representative micrographs of SH-SY5Y cell assays at 168 hours that are obtained as described below. FIG. 3 shows the cells after no treatment. FIG. 4 shows treatment of the cells With nerve growth factor (NGF) only. FIG. 5 shows treatment of the cells with NGF and FK506. FIG. 6 shows treatment of the cells with a water-soluble extract of Centella asiatica obtained as describe below. FIGS. 7 and 8 show treatment of the cells with an ethanolic extract of Centella asiatica (labeled “GK2” as described below). FIG. 9 shows treatment of the cells with asiatic acid. FIG. 10 shows treatment of the cells with asiatic acid and a MEK inhibitor PD 098059.

FIG. 11 depicts the results of thin layer chromatography (TLC) analysis of an ethanolic extract of Centella asiatica (labeled “GK2” as described below), asiatic acid (AA), madecassic acid, madecassoside, and several Centella asiatica fractions (F3-F13). TLC conditions: Kieselgel F254 silica plate (0.25 mm) on alumina developed with chloroform:acetic acid:methanol:water 60:32:12:8, sprayed with anisaldehyde reagent (Wagner and Bladt, 1996, Plant Drug Analysis. A thin layer chromatography atlas. 2nd Ed. Springer-Verlag, Berlin Heidelberg, p. 307) and heated at 100° C. before visualization.

FIG. 12 depicts the results of a high performance liquid chromatography analysis (HPLC) of an ethanolic extract of Centella asiatica (labeled “GK2” as described below), asiatic acid (AA), and several Centella asiatica fractions (F4, F10 and F12). HPLC conditions: A water:acetonitrile gradient with 1% acetic acid on an Econosil 5μ C18 column (250 mm×4.6 mm), detection wavelength 205 nm.

FIG. 13 is bar graph showing mean functional recovery scores for rats given water only (n=3) and GK3/4-treated (300 mg/kg/day) rats.

FIG. 14 shows representative rat footprints at 18 days following axotomy from water-treated (A) or oral administration of GK3/4 at a dose of 300 mg/kg (B). The footprint from the GK3/4-treated rat demonstrates a larger toe spread distance and a smaller heel imprint compared to the control animal.

FIG. 15 is a bar graph showing mean values toe spread distances (first and fifth digits) from water-treated and GK3/4-treated (300 mg/kg/day) rats. For each animal, three footprints were measured from both left and right hind legs and averaged to obtain one value per rat. GK3/4-treated rats exhibit significantly larger values compared to controls.

FIGS. 16A and 16B are light micrographs of axons from a water-treated rat (16A) and a rat given GK3/4 at a dose of 300 mg/kg (16B (magnification×600). Oral administration of GK3/4 elicits larger sized, more myelinated regenerating axons in the distal tibial nerve at 18 days following nerve crush.

FIG. 17 is a bar graph showing the effect of an ethanolic extract (GK7) and four components of Centella asiatica—asiatic acid (AA), asiaticoside (AS), madecassic acid (MA) and madecassoside (MS) on neurite elongation of SH-SY-5Y cells in the presence of nerve growth factor (NGF). Controls were no treatment (NT) and NGF. *P<0.05 compared to NGF alone.

FIG. 18 is a bar graph showing the protective effect of GK (an ethanolic extract of Centella asiatica) against hydrogen peroxide induced toxicity in SHSY5Y neuroblastoma cells in vitro. The effect of GK is dependent on dose, with 100 μg/ml being more effective than 50 μg/ml. GKW, a water extract of the herb was not protective at 200 μg/ml, whereas EGCG was.

### DETAILED DESCRIPTION OF SEVERAL EXAMPLES

For ease of understanding, the following terms used herein are described below in more detail:

“Administration of” and “administering a” compound or composition should be understood to mean providing a compound, a prodrug of a compound, or a pharmaceutical composition as described herein.

“Anti-oxidant neuroprotection” refers to administering anti-oxidants that can scavenge oxidative radicals, or activate anti-oxidant pathways, to reduce the risk of tissue damage (including cell damage) and provide neuroprotection. Oxidative stress has been shown, to a varying degree, to play an important role in the pathogenesis of a number of neurodegenerative situations, including ischemia (stroke), amyotrophic lateral sclerosis (ALS), multiple sclerosis (MS), and Alzheimer's disease. There are a number of oxidative species in the body including hydroxy and hydroperoxy radicals. These radicals cause damage to a number of tissues including nerve cells. There is evidence that damage occurs in the presence of high levels of these agents, or low levels of protective mechanisms such as the enzymes catalase or superoxide dismutase.

An “animal” is a living multicellular vertebrate organism, a category that includes, for example, mammals and birds. A “mammal” includes both human and non-human mammals. “Subject” includes both human and animal subjects.

“Axonal growth” or “axonal regeneration” as used herein refer both to the ability of an axon to grow and to the ability of an axon to sprout. An axon sprout is defined as a new process that extends from an existing or growing axon. (See, e.g., Ma et al., Nat. Neurosci. 2:24-30 (1999)).

“Dosage” means the amount delivered in vivo to a subject of a compound, a prodrug of a compound, or a pharmaceutical composition as described herein.

“Nerve” encompasses a single bundle of nerve fibers or a plurality of bundles of nerve fibers.

“Nerve regeneration” refers to axonal regeneration and restoration of connectivity within neural networks after nerve injury or damage. For example, nerve regeneration may include complete axonal nerve regeneration, including vascularization and reformation of the myelin sheath. More specifically, when a nerve is severed, a gap is formed between the proximal and distal portions of the injured nerve. In order for the nerve axon to regenerate and reestablish nerve function, it must navigate and bridge the gap. Nerve regeneration involves the proximal end forming neurite growth cones that navigate the gap and enter endoneural tubes on the distal portion, thus re-connecting the neural network. It follows that an effective nerve regeneration-promoting agent should promote neurite elongation and should increase the rate of neurite elongation. Hence, an effective nerve regenerating-promoting agent requires a more complex set of activities beyond solely neurite outgrowth potentiating activity. In certain examples, the desirable neurite elongation is significantly greater than that achieved with nerve growth factor alone in cell cultures as described below. For instance, the neurite elongation may be at least about 200 μm, and more particularly about 200 μm to about 1000 μm, in treated cells at 168 hours. With respect to nerve regeneration in animals, a functional improvement may be observed, for example, with at least about a 15% increase in the rate of neurite elongation, more particularly at least about a 30% rate increase, relative to the rate of neurite elongation for untreated nerve injuries.

“Pharmaceutically acceptable salts” include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002).

“Therapeutically-active” refers to an agent, compound or composition capable of inducing a desired therapeutic or prophylactic effect when properly administered to a subject. In this case, the desired therapeutic effect is nerve regeneration and/or anti-oxidant neuroprotection.

“Therapeutically-effective amount” or “nerve regeneration promoting-amount” is an amount sufficient to achieve a statistically significant promotion of nerve cell regeneration compared to a control. Nerve cell regeneration can be readily assessed using an in vitro assay, e.g., the assay described in the Examples below. Alternatively, nerve regeneration can be determined in an in vivo assay or by direct or indirect signs of nerve cell regeneration in a patient. Preferably, the increase in nerve regeneration is at least 10%, preferably at least 30%, and most preferably 50% or more compared to a control.

Alternatively, “therapeutically-effective amount” or “anti-oxidant neuroprotection promoting-amount” is an amount sufficient to achieve a statistically significant reduction in oxidative-mediated nerve cell toxicity. Nerve cell toxicity can be readily assessed using an in vitro assay, e.g., the assay described in the Examples below. Alternatively, nerve cell toxicity can be determined in an in vivo assay or by direct or indirect signs of nerve cell toxicity in a patient.

The above term descriptions are provided solely to aid the reader, and should not be construed to have a scope less than that understood by a person of ordinary skill in the art or as limiting the scope of the appended claims.

The singular terms “a,” “an,” and “the” include plural referents unless context clearly indicates otherwise. Similarly, the word “or” is intended to include “and” unless the context clearly indicates otherwise. The word “comprises” indicates “includes.” It is further to be understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting. All chemical compounds disclosed herein include both the (+) and (−) stereoisomers (as well as either the (+) or (−) stereoisomer), and any tautomers thereof. An analog is a molecule that differs in chemical structure from a parent compound, for example a homolog (differing by an increment in the chemical structure, such as a difference in the length of an alkyl chain), a molecular fragment, a structure that differs by one or more functional groups, or a change in ionization. Structural analogs are often found using quantitative structure activity relationships (QSAR), with techniques such as those disclosed in Remington: The Science and Practice of Pharmacology, 19^{th} Edition (1995), chapter 28. A derivative is a biologically active molecule derived from the base structure.

The compositions and methods disclosed herein may be useful whenever nerve regeneration is sought, for example following any acute or chronic nervous system injury resulting from physical transection/trauma, contusion/compression or surgical lesion, vascular pharmacologic insults including hemorrhagic or ischemic damage, or from neurodegenerative or other neurological diseases. The methods can also be used in association with procedures such as a surgical nerve graft, or other implantation of neurological tissue, to promote healing of the graft or implant, and promote incorporation of the graft or implant into adjacent tissue. According to another aspect, the compositions could be coated or otherwise incorporated into a device or biomechanical structure designed to promote nerve regeneration.

More particularly, pharmaceutical compositions including Centella asiatica plant material, or the extract fraction(s) disclosed herein or components thereof, can be periodically administered to a mammalian patient (e.g., a human patient), in need of such treatment, to promote neuronal regeneration and functional recovery and to stimulate neurite outgrowth and thereby to treat various neuropathological states, including damage to peripheral nerves and the central nervous system caused by physical injury (e.g., spinal cord injury; trauma, sciatic or facial nerve lesion or injury; severed appendage), disease (e.g., diabetic neuropathy), cancer chemotherapy (e.g., by vinca alkaloids and doxorubicin), brain damage associated with stroke and ischemia associated with stroke, and neurological disorders including, but not limited to, various peripheral neuropathic and neurological disorders related to neurodegeneration including, but not limited to: trigeminal neuralgia, glossopharyngeal neuralgia, Bell's palsy, myasthenia gravis, muscular dystrophy, amyotrophic lateral sclerosis, progressive muscular atrophy, progressive bulbar inherited muscular atrophy, herniated, ruptured or prolapsed vertebral disk syndromes, cervical spondylosis, plexus disorders, thoracic outlet destruction syndromes, peripheral neuropathies such as those caused by lead, acrylamides, gamma-diketones (glue-sniffer's neuropathy), carbon disulfide, dapsone, ticks, porphyria, Gullain-Barre syndrome, Alzheimer's disease, Parkinson's disease, and Huntington's chorea. The Centella asiatica plant material or extract fraction(s) are particularly useful for substantially complete axonal nerve regeneration, including vascularization and reformation of the myelin sheath, of a transected nerve of the peripheral nervous system in which the transection was caused by a trauma such as an accidental or intentional severing of the nerve. Such regeneration restores neural connectivity of the transected nerve.

The process for producing the extract fraction generally involves extracting dried Centella asiatica plant material with a solvent, concentrating or removing the solvent to obtain an extract, and then fractionating the extract. Production of the extract and subsequent fractionation results in select extract fraction compositions that include a concentrate of therapeutically-active substances. As mentioned above, one of the therapeutically-active substances is asiatic acid. According to certain illustrative examples, the therapeutically-active extract fraction concentrates may include at least about 0.01, particularly at least about 5, and more particularly at least about 0.05, dry wt. % asiatic acid, based on the total weight of the extract fraction concentrates. In one example, the asiatic acid may be present in an amount of about 6 to about 40 dry wt. %, based on the total dry weight of the extract function. As described below in further detail, asiaticoside and madecassic acid have also been identified as two compounds in the extracts that exhibit activity for nerve regeneration. The extract fractions may be further purified to concentrate the therapeutically-active compounds as described below in more detail. For example, a pharmaceutical composition can be produced that includes at least one extract fraction of Centella asiatica, wherein the extract fraction comprises at least about 0.5 dry wt. %, particularly at least about 5 dry wt. %, and more particularly at least about 8 dry wt. %, of nerve regeneration-active compounds, based on the total dry weight of the extract function.

All parts of the Centella asiatica plant may be used as the raw material for preparing the extracts. More particularly, the plant material may consist of any portion of the plant which contains useful amounts of the therapeutically-active components, which may vary depending on the species, stage of growth, season, and agronomic conditions. According to a specific example, the aerial (above ground) parts of the plant are used. The plant material may be dried by simple exposure to the atmosphere, by forced-air drying (with or without heating) or by freeze-drying. According to one example, the drying is continued until the plant material contains less than about 20 wt. % water, more particularly less than about 5 wt. % water. The compositions disclosed herein may, or may not, include un-separated plant material from Centella asiatica. It may be convenient to utilize as the raw material Centella asiatica materials that already exist in appropriate form and which are generally available as traditional herbs.

The extract may be produced by any suitable method. For example, the extraction may be performed with water, dilute acids, certain organic solvents, including mixtures thereof with water, or supercritical fluids (e.g., supercritical carbon dioxide) followed by drying on a carrier or drying without a carrier. Illustrative extract solvents include alkanols such as methanol or ethanol, mixtures of methanol or ethanol with water, chloroform or hexane. The extraction can occur at any temperature such as, for example, about 10 to about 150, more particularly about 60 to about 90° C., and may be continued for the appropriate time to obtain the desired amount of extract concentrate. The drying carrier material may be un-concentrated Centella asiatica material, maltodextrins, starch, protein, adsorbents or other carrier material. The Centella asiatica material may also be extracted and concentrated without drying to give a liquid extract. The liquid extract may be further diluted with glycerin to provide a “glycerite”.

The extract residue may then be fractionated by any suitable method such as chromatography, liquid-liquid extraction or solid-phase extraction. Illustrative chromatography methods include column chromatography with silica gel, florosil, silicic acid, octadecyl silica, polyamide, ion exchange materials, and mixtures thereof. The chromatography may be performed with a series of successive eluants including water, dilute acids or alkalis, certain organic solvents, or supercritical fluids. Illustrative eluants include alkanes (e.g., hexane), chloroform, esters (e.g., ethylacetate), alkanols (e.g., methanol, ethanol, butanol), acetone, acetonitrile, tetrahydrofuran or aqueous buffer solutions. The fractionation can occur at any temperature such as, for example, about 4 to about 100, more particularly about 18 to about 30° C., and may be continued for the appropriate time to obtain the desired amount of extract fraction concentrate.

The extract fractions may be subjected to further processing for identifying and purifying additional therapeutically-active compounds. For example, Centella asiatica may be extracted with ethanol as described above and in the example below. The extracts then could be dried using rotary evaporation and a centrifugal evaporator. Flavonoid glycosides may be extracted using methanol and the aglycones can be obtained by treating this extract with 1.2N hydrochloric acid (90° C.) and partitioning into ethylacetate. All extracts may be labeled with a unique number to allow tracking. Extracts would be profiled by TLC (thin layer chromatography) and HPLC (high performance liquid chromatography). Column chromatography may be used to fractionate the complex mixture of chemicals found in Centella asiatica extracts. Fractionation of extracts would involve normal phase, reversed phase or polyamide (for flavonoids) stationary phases using VLC for crude fractionation, flash column chromatography for finer separations and preparative TLC or preparative HPLC for compound isolation and purification using standard methods (Houghton et al., Laboratory Handbook for the Fractionation of Natural Extracts (1998)) and specific HPLC separations for components of Centella asiatica (Inamdar et al., Determination of biologically active constituents in Centella asiatica, J. Chromatog. A:127 (1996), Schaneberg et al., An improved HPLC method for quantitative determination of six triterpenes in Centella asiatica extracts and commercial products, Pharmazie 58:381-384 (2003)). The identity of compounds isolated may be determined initially by comparison of chromatographic (TLC, HPLC) and spectroscopic (ultra-violet visible (UV-VIS) spectroscopy and mass spectrometry (MS)) data to known reference compounds. Stand-alone or HPLC-linked spectrometers could be used. Electrospray MS characterization of triterpenes of Centella asiatica has previously been reported (Mauri et al, Electrospray characterization of selected medicinal plant extracts, J Pharm Boimed Anal 23:61-68 (2000)) and other spectroscopic data of known compounds can be obtained from the literature. For novel compounds, UV-VIS spectra and MS can determine the presence of chromophores and molecular weight, respectively. Infra-red (IR) spectroscopy can provide functional group information and most importantly, 1-D and 2-D proton and carbon-13 nuclear magnetic resonance (NMR) spectroscopy can be used for total structure determination. Polarimetry may be used for chiral molecules to determine stereochemistry (if reference data is available) or simply to characterize the compound. Quantitative HPLC analytical protocols can be developed to assess the concentration of known components. The method of normalization (peak area of each component expressed as % of total areas) can be used for unknowns. Changes in the relative concentration of components would be monitored regularly (at least every 3 months); materials showing greater than 10% change will be deemed to have decomposed.

When prepared as an extract, the Centella asiatica extract is preferably dried so that it may be given in the form of tablets, capsules, powders or other convenient form as described in more detail below, or it may be admixed with foods or special food products, or it may be given in the form of a tea or tisane. When prepared as a liquid extract, the Centella asiatica extract may be consumed as drops, or from an appropriate liquid measure (teaspoon), or it may be admixed with other liquids or incorporated into solid food products.

Alternatively, Centella asiatica plant material may be shredded and/or comminuted and administered in the form of a capsule or tablet without first preparing an extract. Such capsules or tablets that include comminuted Centella asiatica plant material may be formulated as described herein.

The Centella asiatica plant material, or extract fraction(s) or components thereof, are administered in a therapeutically effective amount. The therapeutically effective amount will vary depending on the particular agent used and the route of administration. The concentration of therapeutically-active compound or component in the pharmaceutical composition will depend on absorption, inactivation, and excretion rates of the therapeutically-active compound or component, the dosage schedule, and amount administered as well as other factors known to those of skill in the art. It also should be apparent to one skilled in the art that the exact dosage and frequency of administration will depend on the particular compounds administered, the particular condition being treated, the severity of the condition being treated, the age, weight, general physical condition of the particular patient, other medication the individual may be taking

According to a particular example, the Centella asiatica plant material, or extract fraction(s) or a component or compound thereof, may be administered in a dose of at least about 10, particularly 500, and more particularly about 1000, mg/day. The maximum dosage, for example, may be about 10,000, particularly about 5000, and more particularly about 1000 mg/day.

The dose may be a single dose per day, it may be divided into at least two unit dosages for administration over a 24-hour period, or it may be a single continuous dose for a longer period of time, such as 1-10 weeks. Treatment may be continued as long as necessary to achieve the desired results. For instance, treatment may continue for about 3 or 4 weeks up to about 12-24 months.

The therapeutically-active extract fractions disclosed herein can be formulated into therapeutically-active pharmaceutical concentrates or pharmaceutical compositions. The pharmaceutical concentrates or compositions may be administered to a subject parenterally or orally. Parenteral administration routes include, but are not limited to, subcutaneous injections (SQ and depot SQ), intravenous (IV), intramuscular (IM and depot IM), intrasternal injection or infusion techniques, intranasal (inhalation), intrathecal, transdermal, topical, and ophthalmic.

The extract fraction(s) or a component or compound thereof, may be mixed or combined with a suitable pharmaceutically acceptable carrier to prepare pharmaceutical compositions. Pharmaceutically acceptable carriers include, but are not limited to, ion exchangers, alumina, aluminum stearate, lecithin, serum proteins (such as human serum albumn), buffers (such as phosphates), glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat, for example. Liposomal suspensions may also be suitable as pharmaceutically acceptable carriers. Upon mixing or addition of the agent(s), the resulting mixture may be a solution, suspension, emulsion, or the like. These may be prepared according to methods known to those skilled in the art. The form of the resulting mixture depends upon a number of factors, including the intended mode of administration and the solubility of the agent in the selected carrier or vehicle. The effective concentration is sufficient for lessening or ameliorating at least one symptom of the disease, disorder, or condition treated and may be empirically determined.

Pharmaceutical carriers or vehicles suitable for administration of the extract fraction(s) include any such carriers known to be suitable for the particular mode of administration. In addition, the active materials can also be mixed with other active materials that do not impair the desired action, or with materials that supplement the desired action, or have another action. The agents may be formulated as the sole pharmaceutically active ingredient in the composition or may be combined with other active ingredients.

Methods for solubilizing may be used where the agents exhibit insufficient solubility in a carrier. Such methods are known and include, but are not limited to, using cosolvents such as dimethylsulfoxide (DMSO), using surfactants such as Tween®, and dissolution in aqueous sodium bicarbonate.

The extract fraction(s), or components thereof, may be prepared with carriers that protect them against rapid elimination from the body, such as time-release formulations or coatings. Such carriers include controlled release formulations, such as, but not limited to, microencapsulated delivery systems. The therapeutically-active substance is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated condition.

Injectable solutions or suspensions may be formulated, using suitable non-toxic, parenterally-acceptable diluents or solvents, such as mannitol; 1,3-butanediol; water; saline solution; Ringer's solution or isotonic sodium chloride solution; or suitable dispersing or wetting and suspending agents, such as sterile, bland, fixed oils, including synthetic mono- or diglycerides, and fatty acids, including oleic acid; a naturally occurring vegetable oil such as sesame oil, coconut oil, peanut oil, cottonseed oil, and the like; polyethylene glycol; glycerine; propylene glycol; or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

For topical application, the extract fraction may be made up into a solution, suspension, cream, lotion, or ointment in a suitable aqueous or non-aqueous vehicle. Additives may also be included, e.g., buffers such as sodium metabisulphite or disodium edeate; preservatives such as bactericidal and fungicidal agents, including phenyl mercuric acetate or nitrate, benzalkonium chloride or chlorhexidine, and thickening agents, such as hypromellose.

If the extract fraction(s), or components thereof, are administered orally as a suspension, the pharmaceutical compositions may be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. Oral liquid preparations can contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents. The pharmaceutical compositions also may be administered in the form of a tea.

As immediate release tablets, these compositions may contain microcrystalline cellulose, dicalcium phosphate, starch, magnesium stearate and lactose and/or other excipients, binders, extenders, disintegrants, diluents and lubricants.

If oral administration is desired, the Centella asiatica plant material or extract fraction(s) should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, sorbitol, polyvinylpyrrolidone or gelatin; a filler such as microcrystalline cellulose, starch, calcium phosphate, glycine or lactose; a disintegrating agent such as, but not limited to, alginic acid and corn starch; a lubricant such as, but not limited to, magnesium stearate, talc, polyethylene glycol, or silica; a gildant, such as, but not limited to, colloidal silicon dioxide; a sweetening agent such as sucrose or saccharin; disintegrants such as potato starch; and dispersing or wetting agents such as sodium lauryl sulfate; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose or glycerin as a sweetening agent and certain preservatives, dyes and colorings, and flavors. When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily.

The Centella asiatica plant material, or extract fraction(s) or components thereof, may optionally be co-administered with at least one other neurotrophic agent such as nerve growth factor (NGF), FK506, an FKBP12-binding FK506 analog, NGF, IGF-1, aFGF, bFGF, PDGF, BDNF, CNTF, GDNF, NT-3, and NT 4/5, or other herbal extracts such as, for example, ginseng.

In one variant, a transection of a peripheral nerve or a spinal cord injury can be treated by administering a nerve regenerative stimulating amount of the extract fraction(s) or a component or compound thereof, to the mammal and grafting to the peripheral nerve or spinal cord an allograft (Osawa et al., J. Neurocytol. 19:833-849, 1990; Buttemeyer et al., Ann. Plastic Surgery 35:396-401, 1995) or an artificial nerve graft (Madison and Archibald, Exp. Neurol. 128:266-275, 1994; Wells et al., Exp. Neurol. 146:395-402, 1997). The space between the transected ends of the peripheral nerve or spinal cord is preferably filled with a non-cellular gap-filling material such as collagen, methyl cellulose, etc., or cell suspensions that promote nerve cell growth, such as Schwann cells (Xu et al., J. Neurocytol. 26:1-16, 1997), olfactory cells, and sheathing cells (Li et al. Science 277:2000-2002, 1997). The extract fraction(s), or components thereof, can be included together with such cellular or non-cellular gap-filling materials.

In a further variant, the extract fraction(s) or a component or compound thereof, preferably is provided to the site of injury in a biocompatible, bioresorbable carrier capable of maintaining the extract fraction(s) at the site and, where necessary, means for directing axonal growth from the proximal to the distal ends of a severed neuron. For example, means for directing axonal growth may be required where nerve regeneration is to be induced over an extended distance, such as greater than 10 mm. Many carriers capable of providing these functions are envisioned. For example, useful carriers include substantially insoluble materials or viscous solutions prepared as disclosed herein comprising laminin, hyaluronic acid or collagen, or other suitable synthetic, biocompatible polymeric materials such as polylactic, polyglycolic or polybutyric acids and/or copolymers thereof. A preferred carrier comprises an extracellular matrix composition derived, for example, from mouse sarcoma cells.

In a certain example, the extract fraction(s) or a component or compound thereof, is disposed in a nerve guidance channel which spans the distance of the damaged pathway. The channel acts both as a protective covering and a physical means for guiding growth of a neurite. Useful channels comprise a biocompatible membrane, which may be tubular in structure, having a dimension sufficient to span the gap in the nerve to be repaired, and having openings adapted to receive severed nerve ends. The membrane may be made of any biocompatible, nonirritating material, such as silicone or a biocompatible polymer, such as polyethylene or polyethylene vinyl acetate. The casing also may be composed of biocompatible, bioresorbable polymers, including, for example, collagen, hyaluronic acid, polylactic, polybutyric, and polyglycolic acids. In a preferred embodiment, the outer surface of the channel is substantially impermeable.

### EXAMPLE 1

### Fractional Extraction of Centella Asiatica

Dried, shredded Centella asiatica aerial parts (also referred to herein as gotu kola) were purchased from Oregon's Wild Harvest (Batch # GOT-10072C-OGA). The identity of the herb was verified by means of visual examination and by comparing its thin layer chromatographic profile with that reported in the literature (Wagner and Bladt, 1996). The material was stored at room temperature in plastic bags until use.

Centella asiatica herb (242.7 g) was extracted by refluxing with ethanol (2 L) for one hour. The initial ethanol extract was drained off, replaced with a fresh ethanol (1 L) and refluxed for an additional 1 hour. The second lot of ethanol was combined with the first and the total extract filtered through Whatman filter paper to remove plant debris. The extract was evaporated to dryness on a rotary film evaporator (rotavap) to yield a dark green residue which was labeled GK2 (9.93 g). A water extract was prepared by refluxing Centella asiatica (120 g) with water (1.5 L) for 2 hr. The filtered extract was freeze-dried to yield a residue (11.5g), which was labeled GKW.

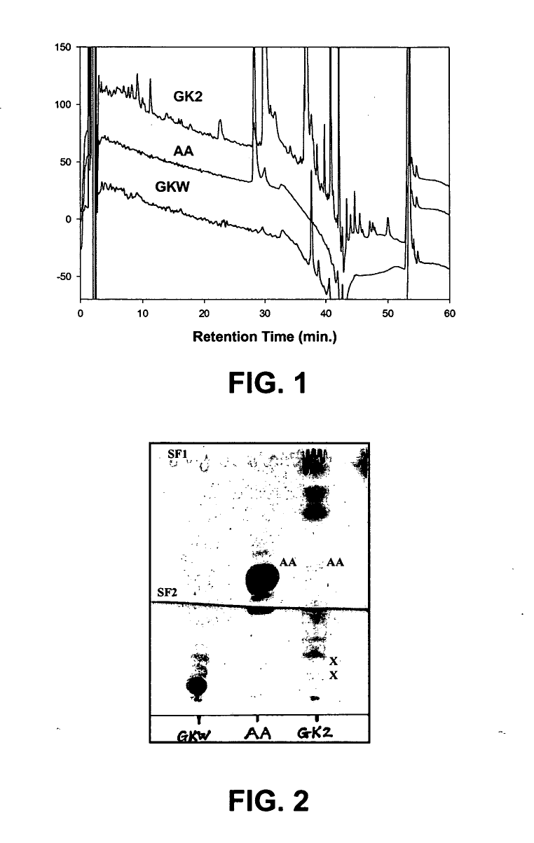

GK2, GKW and a component of Centella asiatica, namely asiatic acid (AA) were compared by thin layer chromatography (TLC) and high performance liquid chromatography (HPLC). The results are shown in FIGS. 1 and 2. In both HPLC and TLC, asiatic acid is present in GK2 but not detectable in GKW. GKW contains mostly very polar compounds as shown by their position near the baseline in TLC (FIG. 2). Polar compounds elute within the first 10 minutes of HPLC so they are not well visualized in FIG. 1. GK2 has a mixture of polar and less-polar components. Compounds common to GKW and GK2 are best visualized on TLC, in the region marked “XX”.

GK2 was fractionated into subfractions using the technique of vacuum liquid chromatography on silica gel. A column (6.5 cm height×9 cm diameter) was prepared in a sintered glass funnel using silica gel 60 (Kieselgel 60; particle size 0.040-0.063 mm, 230-400 mesh). GK2 (4.05 g) was dissolved in ethanol, mixed with a small amount of silica and allowed to dry overnight. The dry GK2/silica mixture was layered over the silica bed in the funnel and then overlaid with a thin (2 mm) layer of fresh silica. The column was eluted with a series of solvents of increasing polarity (see Table 1 below). These were collected separately and evaporated down using a rotavap followed by a speedvac centrifugal evaporator, to yield eleven fractions labeled GKF3 to GKF13 (see Table 1 below).

TABLE 1
Solvents used in fractionation of GK2 extract

| Solvent | Hexane | Chloroform | Methanol | Acetone | Fraction | Weight |
|---|---|---|---|---|---|---|
| number | (mL) | (mL) | (mL) | (ml) | Number | (g) |
| 1 | 300 | | | | GKF3 | 0.28 |
| 2 | 150 | 150 | | | | |
| 3 | 60 | 240 | | | GKF4 | 0.07 |
| 4 | | 300 | | | GKF5 | 0.28 |
| 5 | | 270 | 30 | | GKF6 | 0.74 |
| 6 | | 240 | 60 | | GKF7 | 0.77 |
| 7 | | 210 | 90 | | GKF8 | 0.72 |
| 8 | | 180 | 120 | | GKF9 | 0.17 |
| 9 | | 150 | 150 | | GKF10 | 0.52 |
| 10 | | 120 | 180 | | GKF11 | 0.36 |
| 11 | | 90 | 210 | | GKF12 | 0.24 |
| 12 | | | | 300 | GKF13 | 0.12 |

| | |
|---|---|
| Total weight | 4.27* |

*this weight is greater than the initial amount applied due to the presence of some residual solvent in the fractions.

### EXAMPLE 2

### Neurite Elongation with Centella Asiatica

SH-SY5Y human neuroblastoma cells were maintained in DMEM medium (GIBCO) supplemented with 10% fetal calf serum (SIGMA), 50 IU/mL penicillin, and 50 mg/mL streptomycin (GIBCO) at 37° C. in 7% CO₂. Cells were plated in six-well plates at 1×10⁶ cells/well and treated with 0.4 mM aphidicolin (SIGMA). At five days, cells were washed, treated with nerve growth factor (NGF) (Boehringer Mannheim, Indianapolis, Ind.) at 10 ng/mL (to induce process outgrowth) in the presence or absence of the Centella asiatica extract and extract fractions (100 μg/mL). Media was changed at 96 hours and replaced with fresh media with the agents (NGF plus the Centella asiatica) for an additional 72 hours (total time, 168 hours). All experiments were run in duplicate wells and repeated at least twice for reproducibility.

For analysis of process length, cells (20 fields per well) were randomly photographed at 72 and 168 hours. Neurite lengths were measured on photographic prints using a SummaSketch III digitizing tablet connected with Bioquant Classic 95 software (R&M Biometrics, Nashville, Tenn.); only those processes greater than two times the cell body length were measured. Data from identically treated wells were not different and were therefore combined. Mean values and histograms were constructed from these data. Histograms were compared using a Mann-Whitney U test, which makes no assumptions about the shape of the distribution.

GK2 (100 μg/mL) was found to stimulate neurite outgrowth in human neuronal SH-SY-5Y cells, in the presence of nerve growth factor (NGF), to a greater extent than NGF treatment alone (p<0.05). NGF is used in this in vitro study to differentiate the cells into sympathetic-like neurons. FK506 is employed as a positive control.

Each of the fractions obtained from GK2 was tested (100 μg/mL) for the stimulation of neurite outgrowth in the presence of NGF, yielding the results shown in Table 2 below.

TABLE 2
Effect of Centella asiatica fractions (100 μg/mL) on length of neurite
outgrowth in SK-SH-5Y cells in the presence of NGF

| Test | | | | | GKF5, | | | | |
|---|---|---|---|---|---|---|---|---|---|
| substance | NGF | GK2 | GKF3 | GKF4 | 6, 7, 8, 9* | GKF10 | GKF11 | GKF12 | GKF13 |
| Mean | 115 | 187 | 133 | 202 | toxic, | 158 | 152 | 150 | 169 |
| neurite | | | | | cell | | | | |
| length (μm) | | | | | death | | | | |
| at 168 h | | | | | occurs | | | | |
| Statistical | — | P < 0.05 | P < 0.05 | P < 0.05 | P < 0.05 | P < 0.05 | P < 0.05 | P < 0.05 | P < 0.05 |
| significance | | | | | | | | | |
| of | | | | | | | | | |
| difference | | | | | | | | | |
| to NGF | | | | | | | | | |
| treatment | | | | | | | | | |

*these fractions were tested individually

The above data demonstrate that the active components of the total ethanolic extract of Centella asiatica (GK2) are concentrated significantly in the apolar fraction GKF4. The intermediate fractions GKF5 to GKF9 all proved toxic to the cells. These fractions contained appreciable amounts of chlorophyll. The more polar fractions GKF10 to GKF13 also showed activity, indicating that more than one active component are present.

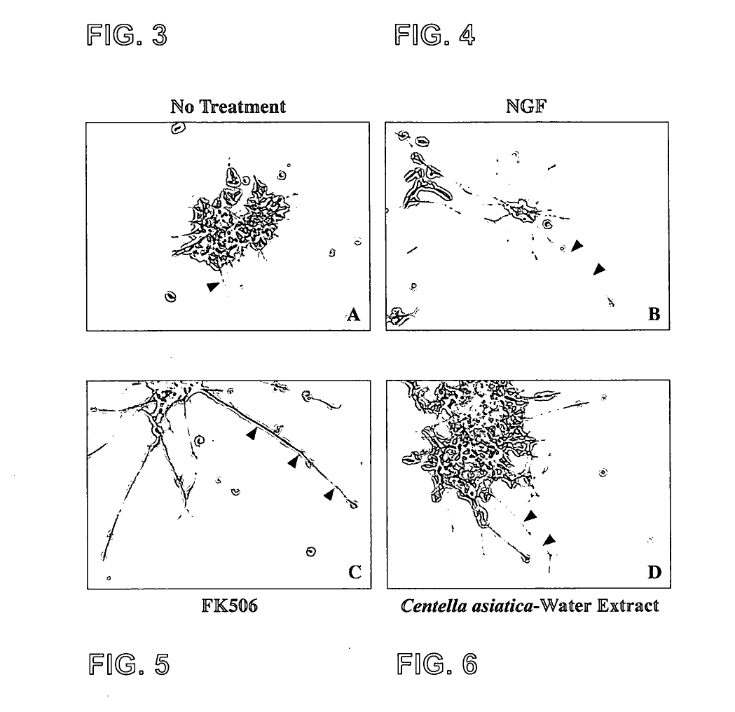

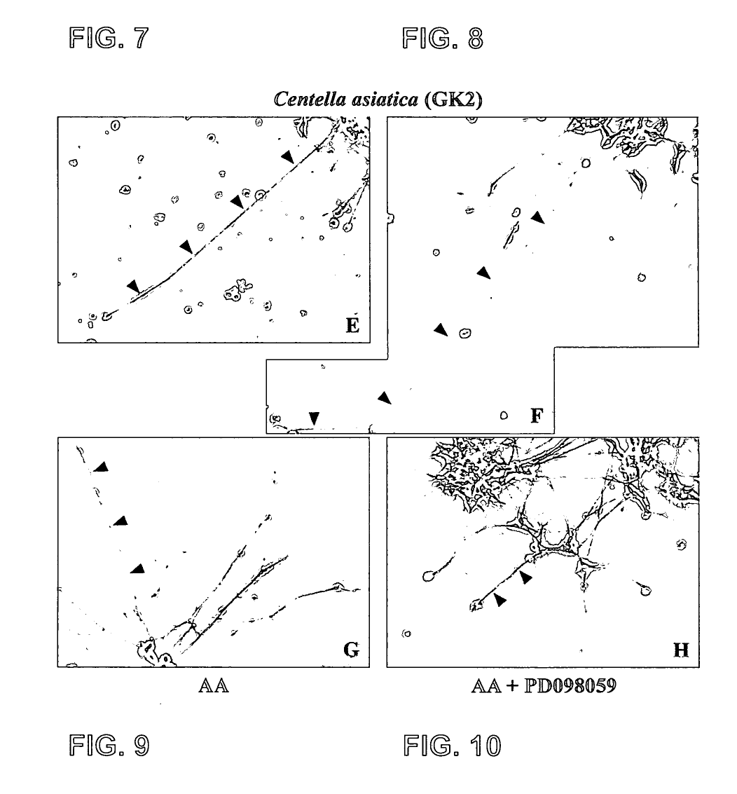

Several micrographs of the SH-SYSY cell assays are included as FIGS. 3-10. Undifferentiated cells exhibit only short processes (see FIG. 3), whereas those differentiated with NGF demonstrate process elongation (see FIG. 4). Elongation is markedly increased with FK506 (see FIG. 5), Centella asiatica (two examples, FIGS. 7 and 8) and AA (see FIG. 9), but not with a water-soluble Centella asiatica extract (see FIG. 6). The activity of AA is prevented by the MEK inhibitor PD 098059 (see FIG. 10). The number of arrowheads in FIGS. 3-10 is indicative of process length.

Thus, the Centella asiatica extract and several of its fractions elicited marked increase in neurite elongation in human SH-SY5Y cells in the presence of NGF to a significantly (p<0.05) greater degree than FK506, a positive control. Like FK506, there was no activity in the absence of NGF. The inactivity of the water-soluble extract of Centella asiatica was consistent with the activity being attributable to non-polar compounds.

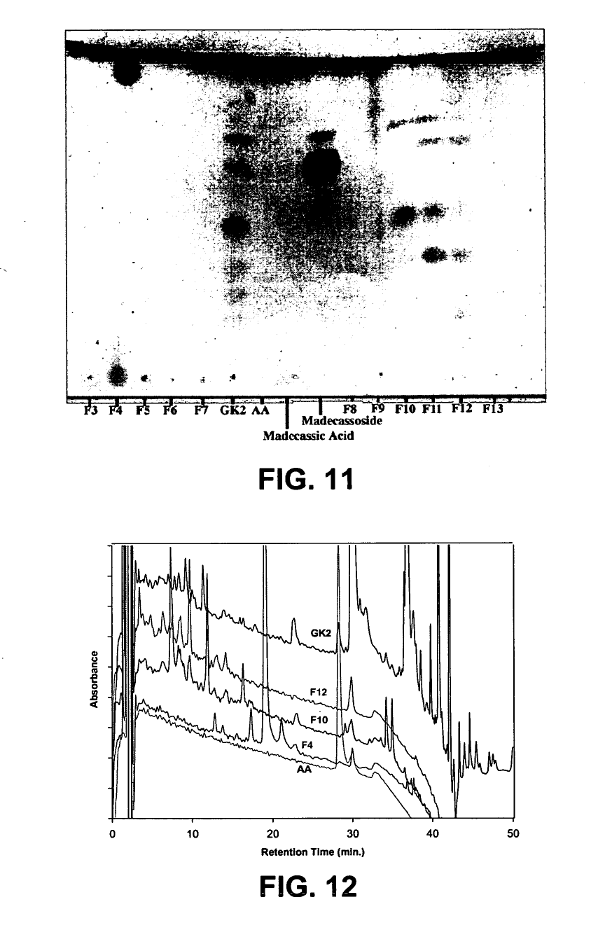

TLC analysis of the fractions is shown in FIG. 11. Asiatic acid (AA) and madecassic acid (MA) elute near the solvent front whereas madecassoside (MS) elutes at a lower R_{f} value. GKF4 and GKF10, 11, 12 and 13 (referred to in FIG. 3 as “F4”, “F10”, “F11”, and “F12” respectively) all promote neurite elongation (Table 2) but vary in their chemical profile. AA and MA may be present in GK2 and GKF4. MS is present in GK2 and GKF12 but not detectable in GKF10.

HPLC analysis of several of the fractions is shown in FIG. 12. AA is present in GK2, in trace amounts in GKF4 but not detectable in GKF10 or GKF12. More specifically, HPLC analysis of the ethanolic extracts GK1 to GK7 (GK1, GK5, GK6 and GK7 refer to ethanolic extracts there were prepared by an identical method to GK2 used in the in vitro studies) of Centella asiatica revealed the presence of asiatic acid, asiaticoside and madecassoside in these extracts, but there were no detectable amounts of madecassic acid in these extracts.

### EXAMPLE 3

### Neurite Elongation with Asiatic Acid

Asiatic acid (AA) is a triterpenoid compound found in Centella asiatica. The activity of asiatic acid in the neurite assay was tested, and asiatic acid was found to show considerable stimulation of neurite outgrowth elongation at a concentration of 1 μM (0.5 μg/mL). Note that this is 0.5% of the concentration of GK2 and of GKF4 which gave a similar effect.

Thin layer chromatographic (TLC) data comparing GK2, AA and GKF4 suggests the possible presence of AA in GK2 and GKF4. The presence of AA in GK2 and GK4 been confirmed by HPLC. AA is not detectable in GK10-13. TLC data shows that GK2, GKF4 and GK10-13 contain substances other than AA. Thus although AA is undoubtedly active in stimulating neurite outgrowth, there are likely a number of other substances in gotu kola that have this effect.

Positive results in SH-SY-5Y cell assays are predictive of successful nerve regeneration in vivo in a sciatic nerve crush model (see Gold, B. G., M. Zeleny-Pooley, M.-S. Wang, P. Chaturvedi, and D. M. Armistead. 1997. A nonimmunosuppressant FKBP-12 ligand increases nerve regeneration. Exp Neurol 147:269-278; Revill, W. P., J. Voda, C. R. Reeves, L. Chung, A. Shirmer, G. Ashley, J. R. Camey, M. Fardis, C. Carreras, Y. Zhou, E. Tucker, D. Robinson, and B. G. Gold. 2002. Genetically engineered analogs of ascomycin for nerve regeneration. J. Pharmacol Exp Therap 302:1278-1285; and Gold, B. G., M. Zeleny-Pooley, P. Chaturvedi, and M.-S. Wang. 1998. Oral administration of a nonimmunosuppressant FKBP-12 ligand speeds nerve regeneration. Neuroreport 9:553-558). The utility of the SH-SY-5Y cell assays as a screening method is further validated by the fact that those compounds demonstrating the greatest in vitro potency in these assays have historically shown the largest increase in nerve regeneration in vivo.

### EXAMPLE 4

### In vivo Assays for Nerve Regeneration

In vivo assays for nerve regeneration are discussed in, for example, Gold et al., Restor. Neurol. Neurosci. 6:287-296, 1994; Gold et al., J. Neurosci. 15:7505-7516, 1995; Wang et al., J. Pharmacol. Exp. Therapeutics 282:1084-1093, 1997; Gold et al., Exp. Neurol. 147:269-278, 1997; Gold et al., Soc. Neurosci. Abst. 23:1131, 1997, which examine the effects of systematic administration of a test compound on nerve regeneration and functional recovery following a crush injury to the rat sciatic nerve. Briefly stated, the right sciatic nerve of anaesthetized rats is exposed, and the nerve crushed twice using forceps at the level of the hip. Following the sciatic nerve crush, the test compound is administered to the rats, e.g., by subcutaneous injection or oral administration. Functional recovery is assessed by determining the number of days following nerve crush until the animal demonstrates onset of an ability to right its foot and move its toes, and the number of days until the animal demonstrates an ability to walk on its hind feet and toes. Nerve regeneration is also assessed by sampling tissues from the sciatic nerve at known (0.5 cm) distances from the crush site and examining the number of myelinated fibers and the size of axons by light microscopy. The axons are also examined by electron microscopy. Axonal areas of both myelinated and unmyelinated fibers are determined by tracing the axolemma using a digitizing tablet connected to a computer with appropriate software. Cumulative histograms are constructed from these data and mean values and standard errors are calculated to assess the effect of administration of the test compound on axonal areas.

To demonstrate in vivo efficacy, a preliminary study was conducted to examine whether oral administration of Centella asiatica (Gotu kola: GK) is able to accelerate nerve regeneration in the sciatic nerve crush model. Six six-week old male Sprague-Dawley rats underwent a bilateral nerve crush at the level of the hip and were given either vehicle (water; n=3) or Centella asiatica extract (GK3 and 4; n=3); extracts GK3 and 4 were prepared by an identical method to GK2 used in the in vitro studies described above, using plant material from the same commercial lot number, and TLC analysis showed that GK2, 3 and 4 had virtually identical profiles. The dried extract was dissolved in the animals' drinking water at a concentration of 2 mg/ml. Based upon the amount of water consumed, the average dose for each animal was calculated to be 300 mg/kg/day over the 18 days of study. Behavioral function and morphological measures were used to assess functional recovery and the animals were perfused with 5% glutaraldehyde at day 18 for histological examination.

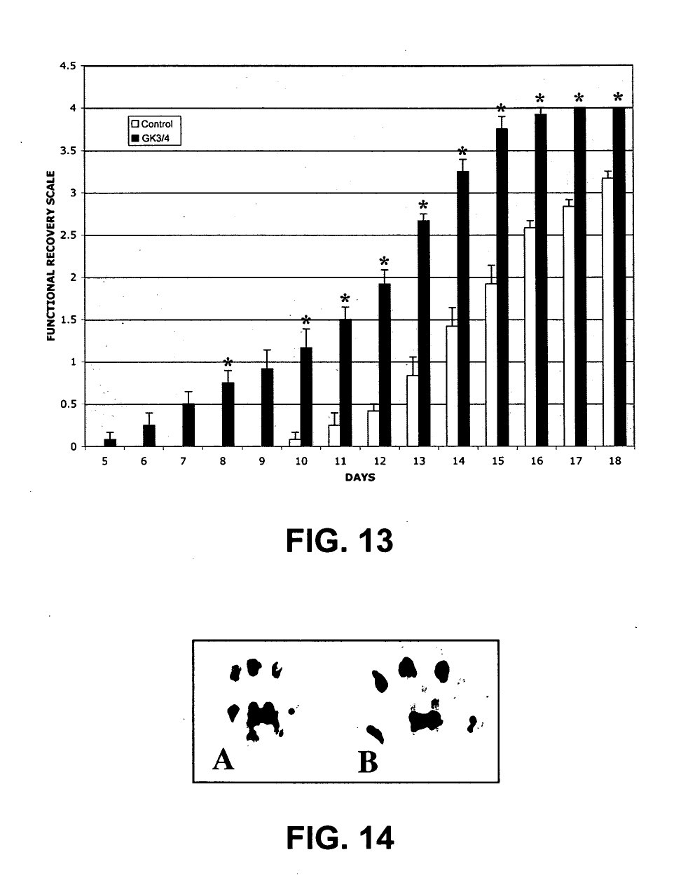

A semi-quantitative scale was used to evaluate functional recovery: 0=complete flaccid paralysis with the foot turned-out upon walking and the toes curved; 1=ability to right the foot and move the toes; 2=ability to constantly walk on the foot; 3=demonstrates toe spread during walking; 4=walks off of heel and shows near normal toe spread. Functional recovery was observed earlier and progressed more rapidly in the GK3/4-treated rats compared to controls with all three GK3/4-treated animals reaching a “4” by day 17 (see FIG. 13).

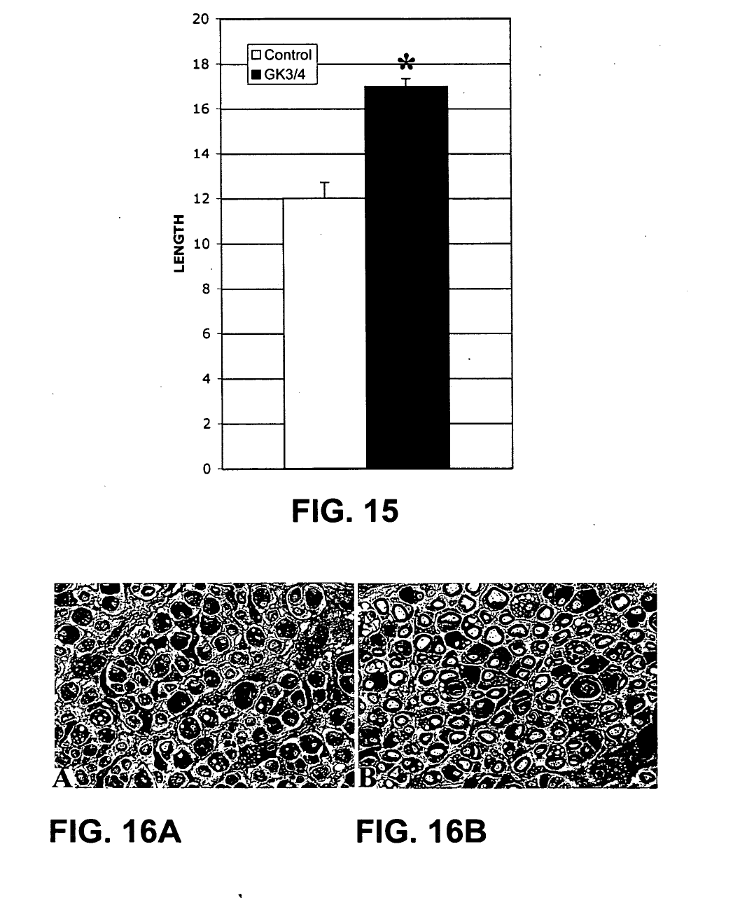

Footprints obtained at 18 days following nerve crush demonstrated a more normal appearance (greater toe spread and less of a heel imprint) compared to vehicle-treated animals (see FIG. 14). The distance between the first and fifth digits was significantly larger in the GK3/4-treated animals compared to controls (see FIG. 15), being close to toe spread distances in uninjured, normal animals (between 18 and 19 mm).

Morphological examination was conducted at 18 days following axotomy. Regenerated axons in the distal tibial nerve branch of the sciatic nerve from GK3/4-treated animals were larger in size and demonstrated more and thicker myelin sheaths compared to controls (see FIGS. 16A and 16B). Thus, regenerating axons in the GK3/4-treated animals were more advanced in their maturation, indicating that the axons arrived in the distal tibial nerve at an earlier time (i.e., grew at a faster rate).

### EXAMPLE 5

### Neurite Elongation with Additional Compounds found in Centella Asiatica

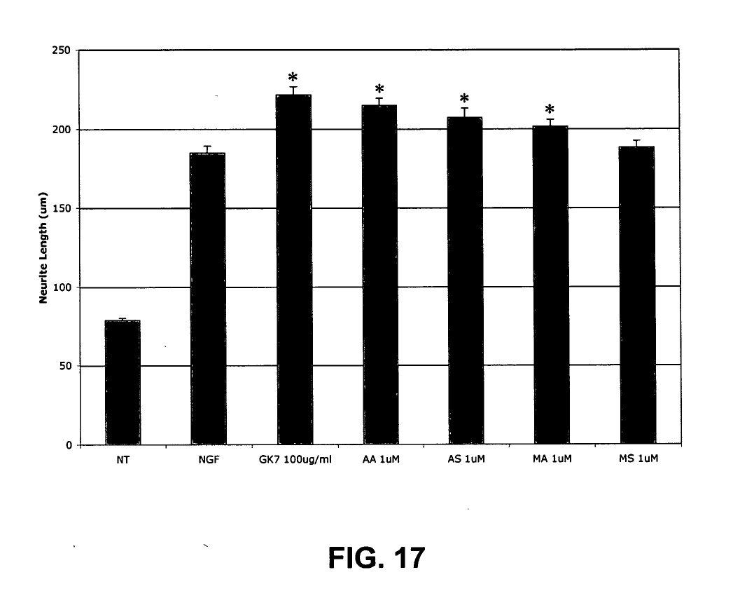

Asiatic acid, asiaticoside, madecassic acid and madecassoside were tested at 1 μm in the neurite elongation assay described above. The results showed that asiatic acid, asiaticoside and madecassic acid were active at this concentration whereas madecassoside as not active (FIG. 17). Although madecassic acid was not detected in the ethanolic extracts, it is possible that is it present at low concentrations, or arises in vivo from hydrolysis of madecassoside. Thus, it is contemplated herein that madecassic acid is an active ingredient derived from the Centella asiatica extracts. In addition, other sources of Centella asiatica plants may well have higher levels of madecassic acid.

### EXAMPLE 6

### Anti-Oxidant Effect of Centella Asiatica Extracts

Dried CA herb was purchased in cut and sift form from Oregon's Wild Harvest, Sandy Oreg. Its identity was verified by comparison of its thin layer chromatography (TLC) profile with that previously reported (Wagner and Bladt, 1996). For the first experiment, CA (32 g) was extracted overnight with cold ethanol (320 ml) followed by refluxing with fresh ethanol (320 ml). The two extracts were combined and evaporated to yield a dark green residue (1.12 g), which was labeled GK1 (GK extract-1). TLC comparison showed no difference in the components extracted by cold or hot ethanol. For the second extraction, Centella asiatica (242.7 g) was extracted by refluxing with ethanol (2 L) for 1 hr. The initial ethanol extract was drained off, replaced with fresh ethanol (1 L) and refluxed for an additional 1 hr. The second lot of ethanol was combined with the first and the total extract filtered through Whatman filter paper to remove plant debris. The extract was evaporated to dryness on a rotary film evaporator (rotavap) to yield a dark green residue (9.93 g), which was labeled GK2 (GK extract-2). Further ethanolic extracts prepared in the same way as GK2 were labeled sequentially up to GK7. These extracts showed virtually identical TLC profiles. CA (120 g) was also refluxed with water (1.5 L) for 2 hr. This extract on freeze-drying yielded a residue (11.5 g), which was labeled GKW1 (GK water extract-1). The extract used for the anti-oxidant experiment was GK7.

SHSY5Y neuroblastoma cells were grown in DMEM/F12 medium (from Gibco) containing 10% fetal calf serum (FCS), 100 ug/ml streptomycin sulphate, 100 U/ml penicillin G in a humidified air/5% CO2 chamber at 37 C. The following method was followed:
Day1. Plate SHSY5Y cells on 24-well plate at 100,000/well
Day4. Remove old medium, add fresh medium with 10 ng/ml NGF.
Day5. Add GK, GKW or EGCG (epigallocatechin gallate; a known antioxidant), overnight.
Day6. Remove old medium (containing test substances), add fresh medium (2% FCS) with 10 ng/ml NGF. Treat cells with or without H₂O₂, for 2-3 hours. Remove medium and H₂O₂, wash 1× with fresh medium with NGF, add fresh medium (2% FCS) with NGF again, incubate overnight.
Day7. Harvest supernatant for LDH assay. Add fresh medium and CellTiter Blue reagent (from Promega, containing resazurin) into each well, incubate for 2 hours, read fluorescence under the Fluorometer. @ 560nm EX/590 nm EM, cut off 590 nm.

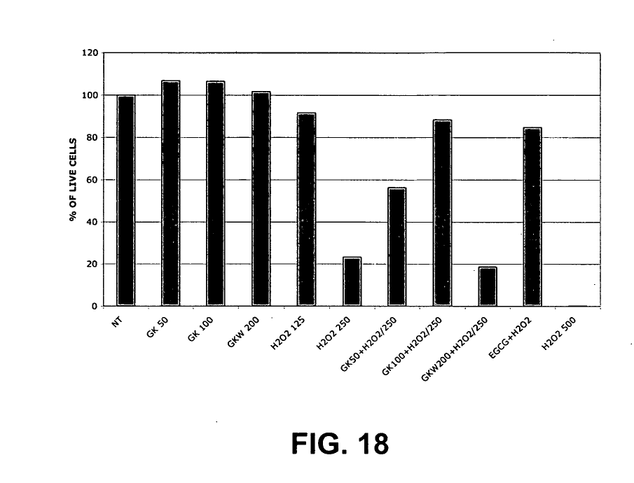

The results are shown in FIG. 18. The FIG. 18 graph shows that H₂O₂ exerts an increasing level of toxicity on SH-SY5Y cells over the concentration range 125 to 500 μM. At 50 and 100 g per ml, preincubation with GK was protective against this toxicity, whereas GKW at 200 μg/ml was not protective. EGCG was also protective.

Having illustrated and described the principles of the disclosed compositions and methods, it will be apparent that these compositions and methods may be modified in arrangement and detail without departing from such principles.

What is claimed is:

1. A method of promoting nerve regeneration in a subject, comprising administering to the subject a therapeutically effective amount of a composition that includes at least one therapeutically-active extract fraction of Centella asiatica.

2. The method of claim 1 wherein the subject has suffered a nerve trauma.

3. The method of claim 2 wherein the subject has undergone reattachment of a severed or partially severed appendage.

4. The method of claim 1 wherein the subject has a disease of the peripheral or central nervous system.

5. The method of claim 1, wherein the subject has at least one partially or fully transected nerve.

6. The method of claim 1, wherein the extract fraction of Centella asiatica is not made with water as the sole extract solvent.

7. The method of claim 1, wherein the extract fraction of Centella asiatica is substantially apolar or semi-polar.

8. The method of claim 1, wherein the extract fraction has the characteristic that it can be fractionated from an ethanolic extract of Centella asiatica via vacuum liquid chromatography on silica gel with a hexane/chloroform eluant.

9. The method of claim 7, wherein the extract fraction of Centella asiatica is substantially apolar.

10. The method of claim 1, wherein the extract of Centella asiatica includes at least about 0.05 dry wt. % asiatic acid based on the total weight of the extract.

11. The method of claim 1, wherein promoting nerve regeneration includes promoting neurite elongation and increasing the rate of neurite elongation.

12. A method of promoting nerve regeneration of at least one partially or fully transected nerve in a mammal, comprising administering to the mammal a therapeutically effective amount of a composition that includes at least one therapeutically-active, substantially apolar extract fraction of Centella asiatica.

13. The method of claim 12, wherein the subject has undergone reattachment of a severed or partially severed appendage.

14. A method of promoting nerve regeneration of at least one partially or fully transected nerve of a peripheral nervous system of a mammal, comprising administering to the mammal a therapeutically effective amount of an extract of Centella asiatica.

15. The method of claim 14, wherein the mammal has undergone reattachment of a severed or partially severed appendage.

16. The method of claim 14, wherein the extract of Centella asiatica is a substantially apolar extract fraction.

17. The method of claim 16, wherein the extract of Centella asiatica includes asiatic acid.

18. The method of claim 17, wherein the extract of Centella asiatica includes at least about 0.01 dry wt. % asiatic acid based on the total weight of the extract.

19. The method of claim 17, wherein the extract of Centella asiatica includes at least about 0.05 dry wt. % asiatic acid based on the total weight of the extract.

20. A method of increasing the rate of neurite elongation of at least one partially or fully transected nerve of a peripheral nervous system of a mammal, comprising administering to the mammal a therapeutically effective amount of asiatic acid, asiaticoside, madecassic acid, any mixture thereof, or a pharmaceutically acceptable salt thereof.

21. The method of claim 20, wherein the asiatic acid, asiaticoside, madecassic acid, any mixture thereof, or the pharmaceutically acceptable salt thereof is administered at a dose of at least about 10 mg/day.

22. A method of increasing the rate of neurite elongation of at least one partially or fully transected nerve of a peripheral nervous system of a mammal, comprising administering to the mammal at least one therapeutically-active extract fraction of Centella asiatica.

23. A method for making an extract fraction of Centella asiatica, comprising:
extracting Centella asiatica plant material resulting in an extract residue; and
successively fractionating the Centella asiatica extract residue with at least two eluants of increasing polarity.

24. The method of claim 23, wherein the extracting is not performed with water as the sole extract solvent.

25. The method of claim 23, wherein the fractionating is performed via chromatography, liquid-liquid extraction, or solid-phase extraction.

26. A pharmaceutical composition comprising an extract fraction of Centella asiatica prepared according to claim 23.

27. The pharmaceutical composition of claim 26, wherein the extract fraction of Centella asiatica is substantially apolar or semi-polar.

28. A method of promoting nerve regeneration of at least one partially or fully transected nerve of a peripheral nervous system of a mammal, comprising administering to the mammal a therapeutically effective amount of Centella asiatica plant material in the form of a tablet or capsule.

29. The method of claim 20, wherein the method comprises administering to the mammal a therapeutically effective amount of asiatic acid or a pharmaceutically acceptable salt thereof.

30. A pharmaceutical composition comprising at least one extract fraction of Centella asiatica, wherein the extract fraction comprises at least about 0.5 dry wt. % of nerve regeneration-active compounds, based on the total dry weight of the extract function.

31. The pharmaceutical composition of claim 30, wherein the nerve regeneration-active compounds comprise asiatic acid, asiaticoside, madecassic acid or any mixture thereof.

32. A method for providing anti-oxidant neuroprotection in a subject comprising administering to the subject a therapeutically effective amount of a composition that includes at least one therapeutically-active extract fraction of Centella asiatica.

33. The method of claim 32, wherein the extract fraction of Centella asiatica is an ethanolic extract.
